(19) United States
(12) Patent Application Publication
(10) Pub. No.: US 2005/0192534 A1
(43) Pub. Date: Sep. 1, 2005

(54) Arrangement for storing, transporting and administering a liquid

(76) Inventors: Peter Wolbring, St. Wendel (DE); Ralph Mosimann, Zuzwil (CH)

Correspondence Address: HARNESS, DICKEY & PIERCE, P.L.C., P.O. BOX 828, BLOOMFIELD HILLS, MI, 48303, US

(21) Appl. No.: 11/062,347

(22) Filed: Feb. 18, 2005

(30) Foreign Application Priority Data
Feb. 20, 2004 (DE) 10 2004 009 918.9

(51) Int. Cl. A61M 5/00

(52) U.S. Cl. 604/111; 604/187

(57) ABSTRACT

An arrangement for storing, transporting and administering a liquid, such as a medical liquid, comprises a syringe barrel. The proximal end of the syringe barrel is closed with a proximal closure element. At the distal end, there is a distal closure element. The two closure elements are connected to one another in a clip-like fashion via a connection element. At least one predetermined breaking point is provided between the two closure elements and the connection element. Thus, a tamper-evident closure for a syringe is formed.

### BACKGROUND OF THE INVENTION

The present invention relates to an arrangement for storing, transporting and administering a liquid. A particularly preferred-application of such an arrangement involves syringes which are pre-filled with a medicament by a pharmaceutical manufacturer, so that the medicament can be administered directly from the “transport package”.

The invention further relates to a tamper-evident closure for a syringe, making it possible for the syringe to be used not just for administering the liquid, but also as an arrangement of the aforementioned type, i.e. for storage and transport.

The production and use of syringes pre-filled by the manufacturer has long been known in the prior art. It is also known to provide syringes with a tamper-evident closure. Thus, EP 0 389 938 A2, for example, discloses a syringe with a syringe barrel at whose distal end there is a needle guard cap. A collar is pushed onto the needle guard cap and its proximal end is adhesively bonded or welded to the syringe barrel. After the collar has been twisted off, it no longer remains by itself on the needle guard cap. The absence of the collar thus indicates that the tamper-evident closure has been opened.

It is also known from this document to arrange a comparable collar also on the proximal end of the syringe barrel. In this way, the inserted plunger rod is secured in its original state. In this case, however, the syringe cannot be pre-filled. In addition, this type of tamper-evident closure is relatively complicated and unwieldy, both in production and also when opening it. Moreover, this tamper-evident closure is relatively easy to manipulate by securing the collar back in its original position with a “hidden” spot of adhesive.

From AT 394 950 B, it is known to design the needle guard cap of a syringe with a base part and a cap part, said base part being connected fixedly to the syringe barrel, and a predetermined breaking point being provided between the base part and the cap part. The cap part moreover has wing-like projections allowing the cap part to be twisted off from the base part. This type of tamper-evident closure requires a special design of the needle guard cap, i.e. it is not possible here to use standardized needle guard caps. In addition, this tamper-evident closure does not provide protection against manipulations at the proximal end of the syringe.

From DE 43 27 171 C1, it is known to provide a “conventional” needle guard cap with a seal which is applied from the outside in free-flowing form onto the adjoining areas of the needle guard cap and of the syringe barrel and then sets. The use of a hot-melt adhesive as seal is proposed in particular. This represents a simple type of tamper-evident closure, but one which is not entirely secure against manipulation, since a comparable hot-melt adhesive can also be easily applied again after the tamper-evident closure has been opened for the first time.

Other tamper-evident closures with which the distal end of a syringe can be secured are known, for example, from DE 197 51 219 A1, DE 39 16 101 A1, DE 199 55 652 A1 or EP 0 440 047 A2. In all these cases, however, the tamper-evident closure involves only the distal end of the syringe, i.e. the proximal end of the syringe remains unsecured.

Securing the proximal end of a syringe by means of a tamper-evident closure is known, for example, from DE 199 25 621 A1. According to said document, a tear-off foil is applied to the proximal opening of the syringe barrel after the plunger stopper has been introduced. In alternative illustrative embodiments, it is proposed to arrange a corresponding foil between the syringe barrel and the plunger stopper, and between the syringe barrel and the plunger rod. For securing at the distal end of the syringe, too, a tear-off foil is proposed which extends between a distal closure cap and the syringe barrel. In principle, a reliable tamper-evident closure can be achieved with such tear-off foils. However, the application of the foils requires suitable packaging machines, which are not always available. Moreover, removal of the tear-off foils may be awkward in some cases.

From DE 100 36 829 A1 it is known to design the proximal end of a syringe barrel with a backstop which prevents inadvertent withdrawal of the plunger stopper from the syringe barrel. In concrete terms, it is proposed, after insertion of the plunger stopper, to narrow the proximal end of the syringe barrel by pressing said syringe barrel in, for example with heated tongs. In this context, reference is made to the fact that this type of backstop at the same time constitutes a tamper-evident closure, but one which only relates to the proximal end of the syringe barrel. In addition, this type of tamper-evident closure may be difficult to integrate into the production sequence employed by a pharmaceutical manufacturer.

Furthermore, for medical syringes, a large number of backstops of different designs are known which are used to avoid inadvertent withdrawal of the plunger stopper from the syringe barrel. As examples of these, reference is made to EP 0 738 517 B1, EP 0 764 450 B1, DE 44 34 644 C2 or WO 94/13339. However, a reliable tamper-evident closure is not associated with these known backstops.

Finally, from EP 0 980 276 B1 or U.S. Pat. No. 4,540,405, it is known to insert a medical syringe, with a syringe barrel made of glass, into an unbreakable retaining body. This is intended to make it possible to press strongly viscous liquids out of the syringe without the danger of the glass barrel being destroyed. However, a tamper-evident closure is not associated with these arrangements.

### SUMMARY OF THE INVENTION

Against this background, it is an object of the present invention to provide an arrangement of the type mentioned at the outset which, on the one hand, can be produced inexpensively and which, on the other hand, permits secure storage and secure transport and also straightforward administration of the liquid. It is a further object of the present invention to provide a tamper-evident closure for a syringe, permitting simple production of the aforementioned arrangement.

According to a first aspect of the invention, the above object is achieved by an arrangement of the type mentioned at the outset, comprising
a syringe barrel for receiving the liquid, said syringe barrel having a proximal end and a distal end,
a proximal closure element which closes the proximal end of the syringe barrel,
a distal closure element which closes the distal end of the syringe barrel, and
a connection element which connects the proximal and distal closure elements to one another in a clip-like fashion,
wherein at least one predetermined breaking point is provided between the two closure elements and the connection element.

According to another aspect, there is provided a tamper-evident closure for syringe, having a proximal closure element designed to be arranged on the proximal end of a syringe barrel, having a distal closure element designed to be arranged on the distal end of the syringe barrel, and having a connection element designed to connect the proximal and distal closure elements to one another in a clip-like fashion, wherein at least one predetermined breaking point is provided between the two closure elements and the connection element.

The present invention thus follows an approach of forming a tamper-evident closure for a syringe by connecting two closure elements at the ends of a syringe barrel via a connection element so that they secure each other in their respective position. To remove only one of the two closure elements from the syringe barrel, an irreversible separation of the two closure elements is necessary, which is favoured by the predetermined breaking point provided. The practical implementation is associated with low expenditure, since the two closure elements for the syringe barrel are generally already present for other reasons. Whereas the two closure elements in the previously known arrangements were secured separately in at least two operations, if indeed at all, it now suffices to provide one common connection element. The novel arrangement is therefore very easy and inexpensive to produce.

In addition, the invention affords a very reliable tamper-evident closure since any manipulation always affects both closure elements. By contrast, in the previously known solutions it has in principle been possible to manipulate the closure at one end of the syringe barrel while the other end remains completely undamaged. Moreover, the solution proposed here results in a tamper-evident closure which is immediately noticeable because the connection element necessarily extends over a considerable part of the syringe barrel and is thus readily apparent from the outside.

Furthermore, the connection element, as will be shown below on the basis of preferred illustrative embodiments, can very easily be realized in such a way that it fits onto standardized or at least widely used closure elements and syringe barrels. Mechanical closure of the arrangement is possible, so that the novel solution can be very easily integrated into widely used and established production processes and is possible in large batch numbers.

Finally, the connection element extending along the syringe barrel provides a simple possibility of protecting the syringe barrel itself from mechanical or other kinds of influences, as will be set out below. The novel arrangement thus not only provides protection against tampering, but also protects the syringe barrel from inadvertent damage.

All in all, the novel arrangement thus permits, in an inexpensive manner, secure transport, storage, and administering of a liquid, which is particularly desirable in the medical sector.

In a preferred embodiment of the invention, the proximal closure element comprises a plunger stopper arranged in the syringe barrel, and a backstop preventing proximal removal of the plunger stopper from the syringe barrel.

In this embodiment, the proximal closure element is realized with the aid of structural parts which are also used for the subsequent administration of the liquid. By this means, the number of parts in the novel arrangement and the novel tamper-evident closure is reduced. The invention can thus be realized even more simply and inexpensively.

In a further embodiment, the backstop is secured as a separate structural part on the proximal end of the syringe barrel.

This embodiment makes it possible to use conventional and thus widely employed syringe barrels for the novel arrangement, as a result of which the production costs are further reduced. Another advantage is the possibility of separating syringe barrel and backstop from one another for subsequent recycling. In this way too, the overall costs can be further reduced.

In a further embodiment, the distal closure element and the connection element are connected integrally to one another.

Typically, when administering the content of the syringe, the distal closure element has to be removed, whether this is a needle guard cap or the distal closure element is replaced by an injection needle. By the distal closure element and the connection element being designed as one piece, the number of operating steps required for administering the content of the syringe is reduced. Administration is simplified in this way. In addition, this embodiment results in a further reduced number of parts, permitting a further reduction in the production costs.

In an alternative embodiment, the distal closure element and the connection element are two separate structural parts, the connection element engaging at least partially around the distal closure element.

Compared to the previous alternative, this embodiment entails a larger number of parts. On the other hand, it has the advantage that the novel arrangement can be designed with standardized closure elements at the distal end of the syringe barrel, which likewise permits a reduction in the production costs. In addition, existing syringes with distal closure elements can be equipped very easily with the novel tamper-evident closure. This embodiment can therefore be very easily integrated into existing production sequences.

In a further embodiment, the connection element surrounds the syringe barrel in a sleeve-like or even pot-like fashion.

In both cases, the connection element protects the syringe barrel against external damage, as a result of which both transport and storage are made easier and more secure. This applies in particular when the syringe barrels are made of glass, which affords advantages in terms of sterilizability.

In a further embodiment, the connection element has at least one window-like recess in the area of the syringe barrel.

Such a recess makes it possible to view the syringe barrel and its content even when the syringe barrel is engaged in the sleeve-like or pot-like connection element. It is thus possible to visually monitor the syringe content without breaking open the tamper-evident closure. In addition, such a recess leads to a weight reduction and thus also to a reduction in the transport, storage and production costs.

In a further embodiment, the connection element has wing-like projections on its outside.

Such projections make it easier to detach the connection element from one or both of the closure elements. This further simplifies and accelerates the administration of the syringe content.

In a further embodiment, the proximal closure element and the connection element are designed as two pieces but are connected to one another in an irreversible manner. The connection of the two structural parts can be done, for example, by adhesive bonding, welding, shrinking, pressing, locking or otherwise.

This embodiment permits particularly simple application and closing of the tamper-evident closure. This applies in particular when the proximal closure element comprises a backstop arranged on the syringe barrel, which backstop is secured on said syringe barrel after the plunger stopper has been inserted. This embodiment permits particularly efficient and straightforward production.

In a particularly preferred embodiment, the proximal closure element and the connection element are connected to one another with a form fit. This includes in particular a press-fit connection, shrink-fit connection and/or locking connection.

This embodiment has the advantage that the tamper-evident closure is closed mechanically, which can be integrated very easily into existing production sequences.

In a further embodiment, the connection element has a ring portion which surrounds the syringe barrel and engages irreversibly in a groove formed on the proximal closure element.

This embodiment is particularly preferred in combination with a backstop as a structural part of the proximal closure element. By means of the ring portion, the backstop can in fact be fixed permanently on the syringe barrel, so that, even after the tamper-evident closure has been broken open, inadvertent withdrawal of the plunger stopper from the syringe barrel is avoided. This is especially advantageous if highly toxic and/or very expensive medicaments are contained in the syringe barrel.

It will be appreciated that the aforementioned features and the features still to be explained below can be used not only in the respectively stated combination, but also in other combinations or singly, without departing from the scope of the present invention.

### BRIEF DESCRIPTION OF THE DRAWINGS

Illustrative embodiments of the invention are shown in the drawing and are explained in greater detail in the description below. In the drawing:

FIG. 1 shows an illustrative embodiment of the novel arrangement in a perspective view,

FIG. 2 shows the illustrative embodiment from FIG. 1, but without the pot-like or sleeve-like connection element used there,

FIG. 3 shows the illustrative embodiment from FIG. 1 in a cross-sectional view, and

FIG. 4 shows a syringe for administering a liquid after the tamper-evident closure from the illustrative embodiment according to FIG. 1 has been broken open.

### DESCRIPTION OF PREFERRED EMBODIMENTS

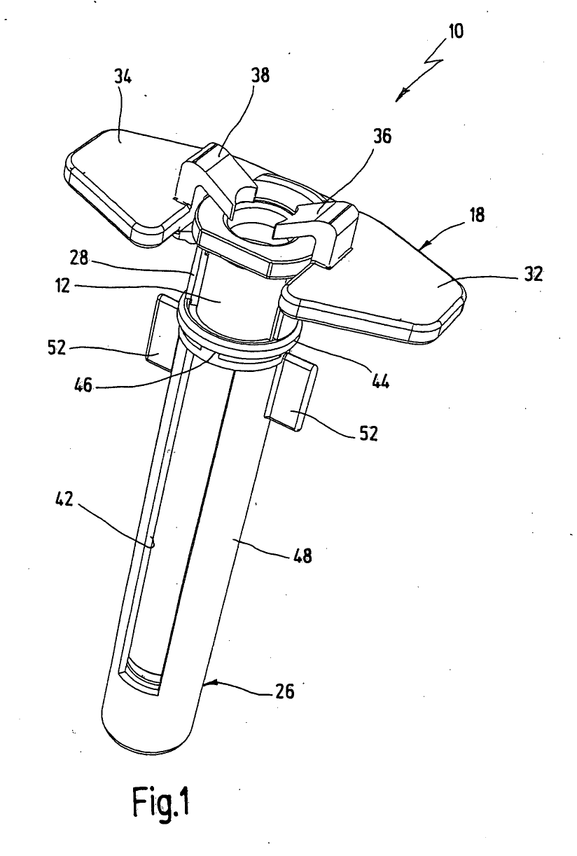

In FIGS. 1 to 3, an illustrative embodiment of an arrangement according to the invention is indicated overall by reference number 10.

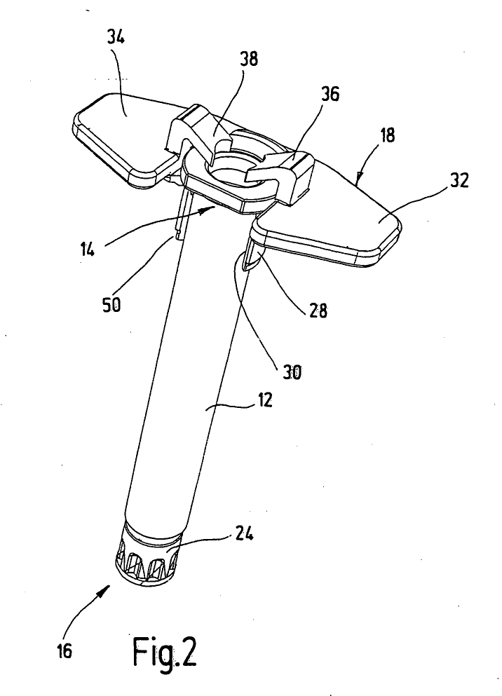

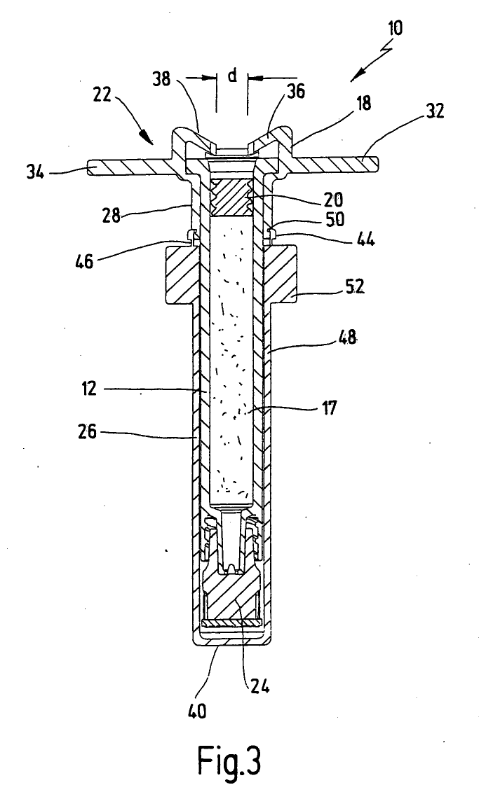

The arrangement 10 comprises a syringe barrel 12 with a proximal end 14 and a distal end 16 (see FIG. 2). A backstop 18 for a plunger stopper 20 (FIG. 3) arranged in the syringe barrel 12 is provided at the proximal end 14. In the illustrative embodiment, backstop 18 and plunger stopper 20 together form a proximal closure element, which in FIG. 3 is indicated overall by reference number 22.

A closure cap 24 known per se is arranged at the distal end 16 of the syringe barrel. Instead of this, however, it would also be possible to fit here, for example, an adapter for a needle, or a needle with a needle guard cap. In addition, the closure cap at the distal end 16 of the syringe barrel 12 can also have a shape other than the one shown here.

Reference number 26 indicates a connection element which has a pot-like configuration in the present illustrative embodiment. It encloses the syringe barrel 12 and the closure cap 24 arranged at its distal end 16 and, in the area of the proximal end 14, it is connected to the backstop 18 in a manner described in more detail below. Together, the proximal closure element 22 (here with backstop 18 and plunger stopper 20), the distal closure element (here the closure cap 24) and the connection element 26 form a tamper-evident closure for the syringe barrel 12.

In this illustrative embodiment, the backstop 18 is a separate structural part which is secured on the proximal end of the syringe barrel 12. For this purpose, the backstop 18 has a wall 28 in the form of a cylinder shell which firmly engages around the syringe barrel 12 in the area of its proximal end 14. A lateral opening area 30 of the wall 28 is dimensioned in such a way that the backstop 18 can be pushed from the side onto the proximal end of the syringe barrel 12 in the manner of a snap-fit connection.

At the upper end of the wall 28, the backstop 18 has two laterally protruding plate areas 32, 34 which form a finger support for the person using the syringe. The backstop 18 shown is thus intended principally for syringe barrels in which a finger support of this kind is not formed on the syringe barrel. In principle, however, the present invention can also be used in such syringe barrels, the backstop 18 then being designed without or with correspondingly modified plate areas 32, 34.

Laterally from the syringe barrel 12, a claw-like catch hook 36, 38 is arranged on each of the two plate areas 32, 34. The catch hooks 36, 38 point radially towards one another. Their free ends are also inclined towards the distal end 16 of the syringe barrel 12. The clear distance between the two catch hooks 36, 38, is indicated by d in FIG. 3. This corresponds approximately to the external diameter of the plunger stopper 20, so that the plunger stopper 20, upon withdrawal from the syringe barrel 12, comes into contact with the catch hooks 36, 38. If the plunger stopper 20 is then withdrawn further from the syringe barrel 12, the clear distance d between the two catch hooks 36, 38 narrows because of the selected inclination. The holding pressure on the plunger stopper 20 increases, so that complete removal of the plunger stopper 20 from the syringe barrel 12 is prevented.

In the presently preferred illustrative embodiment shown, the clear distance d between the two catch hooks 36, 38 is approximately identical to the clear internal diameter of the syringe barrel 12. In other words, the two catch hooks 36, 38 do not jut into a projected circular line corresponding to the internal diameter of the syringe barrel 12. However, since the plunger stopper 20 in the syringe barrel is pressed radially together in order to achieve good lateral sealing on the inside wall of the barrel, the two catch hooks 36, 38 can nevertheless reliably secure the plunger stopper 20. This particular type of configuration of the backstop 18 is the subject of a parallel plate application (filed on the same date and claiming priority from German patent application DE 10 204 009 919.7) assigned to the present Assignee, which is incorporated here by reference. However, it is not essential for realizing the claiming invention set out herein, i.e. the present arrangement can in principle also be realized with other types of backstops, such as are known, for example, from the prior art mentioned in the introduction.

In the preferred illustrative embodiment, the connection element 26 has a pot-like configuration, the syringe barrel 12 engaging with its distal closure cap 24 into the “pot” with a precise fit (FIG. 3). However, in a deviation from this configuration, the connection element 26 can, for example, also be designed without base 40, so that it then instead has the form of a sleeve. In this context, it is important simply that the connection element 26 ensures that the closure cap 24, more generally a closure element arranged at the distal end 16, cannot be removed as long as the connection element 26 sits on the syringe barrel 12. For this purpose, it suffices, for example, to provide a flanged edge, a thickened portion or a bridge formed on the connection element 26 in the area of the closure cap 24.

In the preferred illustrative embodiment, the connection element 26 has two window-like recesses 42 which extend parallel to one another but are offset by 180° to one another on the outer circumference of the connection element 26. Accordingly, the second recess 42 cannot be seen in the views in FIGS. 1 and 3.

At its upper (proximal) end, the connection element 26 has a ring 44 which is connected to the sleeve-like body 48 of the connection element 26 via four thin bridges 46. The bridges 46 are here distributed uniformly on the outer circumference of the connection element 26. They form predetermined breaking points at which the connection element 26 can be broken open and the body can then be detached from the syringe barrel 12.

In the present illustrative embodiment, the ring 44 of the connection element 26 sits with a precise fit in a groove 50 formed at the distal end of the wall 28. Ring 44 and groove 50 form a lock closure with which the connection element 26 and the backstop 18 are joined together. Alternatively and/or in addition, the ring 44 could also be adhesively bonded, welded or otherwise irreversibly connected to the backstop 18.

Reference number 52 indicates two wing-like projections which are formed just below the ring 44 on the connection element 26. The wing-like projections 52 permit simple turning of the connection element 26 relative to the backstop 18, specifically in such a way that the bridges 46 break open. By virtue of the fact that the ring 44 remains in the groove 50 on the backstop 18, said backstop 18 is still secured at the proximal end of the syringe barrel 12, i.e. it cannot be removed without irreversibly destroying the ring 44.

By means of the preferred design of the connection element 26 in the form of a sleeve or pot, the syringe barrel 12 is additionally protected during transport and storage. For realization of the tamper-evident closure, however, this form of the connection element 26 is not absolutely necessary. Particularly in syringe barrels 12 made of plastic, the connection element 26 can also have a completely different shape as long as it satisfies the intended function, namely that of securing the proximal and distal closure elements (here the backstop 18 and the closure cap 24, respectively) in their respective positions. For example, the connection element 26 can be a simple bridge which extends, outside the syringe barrel 12, from the closure cap 24 as far as the backstop 18. The connection element is preferably made of a stiff or at least minimally extensible material, in order to effectively prevent removal of the closure cap and/or of the backstop 18.

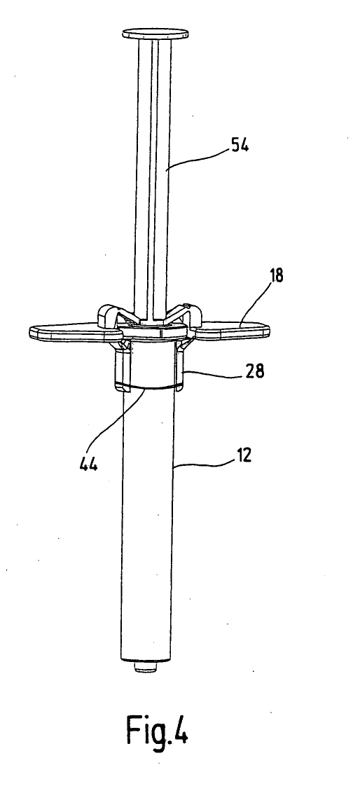

FIG. 4 shows the syringe barrel 12 with the backstop 18 after removal of the connection element 26 and of the closure cap 24 and after a plunger rod 54 has been screwed into the plunger stopper 20. The plunger rod 54 can be supplied to the end user separately from the arrangement 10. Alternatively, it is also possible for the plunger rod 54 to be already screwed into the plunger stopper 20, or otherwise secured in it, by the manufacturer. For the sake of clarity, however, the plunger rod 54 is not shown in FIGS. 1 to 3.

1. An arrangement for storing, transporting and administering a liquid, comprising
a syringe barrel for receiving the liquid, said syringe barrel having a proximal end and a distal end,
a proximal closure element for closing the proximal end of the syringe barrel,
a distal closure element for closing the distal end of the syringe barrel, and
a connection element which connects the proximal and distal closure elements to one another in a clip-like fashion,
wherein at least one predetermined breaking point is provided between the two closure elements and the connection element.

2. The arrangement of claim 1, wherein the proximal closure element comprises a plunger stopper arranged in the syringe barrel, and a backstop preventing proximal removal of the plunger stopper from the syringe barrel.

3. The arrangement of claim 2, wherein the backstop is secured as a separate structural part on the proximal end of the syringe barrel.

4. The arrangement of claim 1, wherein the distal closure element and the connection element are connected integrally to one another.

5. The arrangement of claim 1, wherein the distal closure element and the connection element are two separate structural parts, the connection element engaging at least partially around the distal closure element.

6. The arrangement of claim 5, wherein the connection element surrounds the syringe barrel in a sleeve-like or pot-like fashion.

7. The arrangement of claim 6, wherein the connection element has at least one window-like recess in the area of the syringe barrel.

8. The arrangement of claim 1, wherein the connection element has wing-like projections on its outside.

9. The arrangement of claim 1, wherein the proximal closure element and the connection element are designed as two pieces but are connected to one another in an irreversible manner.

10. The arrangement of claim 9, wherein the proximal closure element and the connection element are connected to one another with a form fit.

11. The arrangement of claim 10, wherein the connection element has a ring portion which surrounds the syringe barrel and engages irreversibly in a groove formed on the proximal closure element.

12. A tamper-evident closure for a syringe barrel having a proximal and a distal end, the tamper-evident closure comprising a proximal closure element designed to be arranged on the proximal end of the syringe barrel, comprising a distal closure element designed to be arranged on the distal end of the syringe barrel, and comprising a connection element designed to connect the proximal and distal closure elements to one another in a clip-like fashion, wherein at least one predetermined breaking point is provided between the two closure elements and the connection element.

13. The tamper-evident closure of claim 12, wherein the proximal closure element comprises a plunger stopper to be arranged in the syringe barrel, and a backstop for preventing proximal removal of the plunger stopper from the syringe barrel.

14. The tamper-evident closure of claim 13, wherein the backstop is designed to be secured as a separate structural part on the proximal end of the syringe barrel.

15. The tamper-evident closure of claim 12, wherein the distal closure element and the connection element are connected integrally to one another.

16. The tamper-evident closure of claim 12, wherein the distal closure element and the connection element are two separate structural parts, the connection element engaging at least partially around the distal closure element.

17. The tamper-evident closure of claim 12, wherein the connection element surrounds the syringe barrel in a sleeve-like or pot-like fashion.

18. The tamper-evident closure of claim 17, wherein the connection element has at least one window-like recess in the area of the syringe barrel.

19. The tamper-evident closure of claim 12, wherein the connection element has wing-like projections on its outside.

20. The tamper-evident closure of claim 12, wherein the proximal closure element and the connection element are designed as two pieces adapted for connection to one another in an irreversible manner.

21. The tamper-evident closure of claim 20, wherein the proximal closure element and the connection element are designed to be connected to one another with a form fit.

22. The tamper-evident closure of claim 21, wherein the connection element has a ring portion adapted to surround the syringe barrel and the proximal closure element has a groove, the ring portion being adapted to engage irreversibly in the groove.
